(19) United States
(12) Patent Application Publication
(10) Pub. No.: US 2002/0123727 A1
(43) Pub. Date: Sep. 5, 2002

(54) Cellulosic product having high compression recovery

(76) Inventors: Richard A. Edmark, Seattle, WA (US); Darryl T. Genest, Federal Way, WA (US)

Correspondence Address: PATENT DEPARTMENT CH2J29, WEYERHAEUSER COMPANY, P.O. BOX 9777, FEDERAL WAY, WA, 98063-9777, US

(21) Appl. No.: 10/002,103

(22) Filed: Nov. 14, 2001

Related U.S. Application Data
(60) Provisional application No. 60/248,519, filed on Nov. 14, 2000.

(51) Int. Cl. A61F 13/15; A61F 13/20

(52) U.S. Cl. 604/366; 604/365; 604/370; 604/372; 604/373; 604/374; 604/375

(57) ABSTRACT

The present invention provides a bonded cellulosic fibrous product having low creep. The product includes crosslinked cellulosic fibers and a bonding agent. Methods for forming the bonded cellulosic fibrous product and personal care absorbent products that include the bonded product are also provided.

### CROSS-REFERENCE TO RELATED APPLICATION

This application claims the benefit of the filing date of U.S. patent application Ser. No. 60/248,519, filed Nov. 14, 2000.

### FIELD OF THE INVENTION

The present invention relates generally to a cellulosic fibrous product and, more particularly, to a cellulosic fibrous product having high compression recovery.

### BACKGROUND OF THE INVENTION

Crosslinked cellulosic fibers are advantageously incorporated into a variety of fibrous products to enhance product bulk, resilience, and dryness. Absorbent articles, such as diapers, are typically formed from fibrous composites that include absorbent fibers such as wood pulp fibers, and can additionally include crosslinked cellulosic fibers. When incorporated into absorbent articles, such fibrous composites can provide a product that offers the advantages of high liquid acquisition rate and high liquid wicking capacity imparted by the absorbent fibers and crosslinked fibers, respectively.

Personal care absorbent products, for example, infant diapers, adult incontinence products, and feminine care products, include liquid acquisition and/or distribution layers that serve to rapidly acquire and then distribute acquired liquid to a storage core for retention. To achieve rapid acquisition and distribution, these layers may include crosslinked cellulosic fibers, which impart bulk and resilience to the layers.

Liquid acquisition composites for use in personal care absorbent products, such as infant diapers, optimally have low densities in the range from about 0.04 to about 0.06 g/cm³. While such low density composites can be manufactured, to economically ship such low density composites to, for example, a diaper manufacturer for incorporation into a diaper, it is first necessary to densify the composite to a density of about 0.2 g/cm³. Unfortunately, low density composites often fail to return to low density after shipping and prior to incorporation into the absorbent product.

Accordingly, there exists a need for a product suitable for use as an acquisition composite that can be manufactured to have a low density, densified for shipping, and then rebound to, or near, its original density for incorporation into an absorbent product. The present invention seeks to fulfill these needs and provides further related advantages.

### SUMMARY OF THE INVENTION

In one aspect, the present invention provides a cellulosic fibrous product having low creep. The product includes crosslinked cellulosic fibers and a bonding agent. The product can optionally include other fibers.

In another aspect of the invention, methods for forming the low creep cellulosic fibrous product is provided. In one embodiment, the product is formed by an airlaid process, or an extrusion process.

In a further aspect, the present invention provides absorbent articles that include the cellulosic fibrous product. The product can be combined with one or more other layers to provide structures that can be incorporated into absorbent articles such as infant diapers, adult incontinence products, and feminine care products.

### DETAILED DESCRIPTION OF THE PREFERRED EMBODIMENT

In one aspect, the present invention provides a cellulosic fibrous product that returns to, or near, its original low density for incorporation into an absorbent article after the product has been densified for shipping. The low creep product includes bonded crosslinked cellulosic fibers. The product possesses the advantageous properties of bulk and resiliency associated with intrafiber crosslinked fibers and the advantage of structural integrity imparted to the structure by the bonding between fibers. The product is a bonded web in which the crosslinked fibers and the bonded structure of the web itself contribute to the resiliency and liquid acquisition performance of the web.

In the present invention, bonding between fibers can be attained by treating the fibers with a bonding agent. In one embodiment, the bonding agent is a latex. In one embodiment, the product is prepared treating crosslinked cellulosic fibers with a latex and a latex fixative.

The product can be formed by (1) forming a web of crosslinked cellulosic fibers; (2) treating the web with a bonding agent; and (3) heating the web at a temperature and for a time sufficient to effect bonding between fibers. The product can be formed by airlaid, wetlaid, foam-forming, and extrusion processes.

The product formed in accordance with the present invention has an initial density in the range of from about 0.02 to about 0.06 g/cm³. The low density product can be densified to a higher density product having a density in the range of from about 0.075 to about 0.2 g/cm³. The densified product returns to, or near, its original density. Products formed in accordance with the invention return to from about 75 to about 100 percent of their original density after 7 days at 0.5 psi.

Any one of a number of crosslinking agents and crosslinking catalysts, if necessary, can be used to provide the product of the invention. The following is a representative list of useful crosslinking agents and catalysts. Each of the patents noted below is expressly incorporated herein by reference in its entirety.

Suitable urea-based crosslinking agents include substituted ureas such as methylolated ureas, methylolated cyclic ureas, methylolated lower alkyl cyclic ureas, methylolated dihydroxy cyclic ureas, dihydroxy cyclic ureas, and lower alkyl substituted cyclic ureas. Specific urea-based crosslinking agents include dimethyldihydroxy urea (DMDHU, 1,3-dimethyl-4,5-dihydroxy-2-imidazolidinone), dimethyloldihydroxy-ethylene urea (DMDHEU, 1,3-dihydroxymethyl-4,5-dihydroxy-2-imidazolidinone), dimethylol urea (DMU, bis[N-hydroxymethyl]urea), dihydroxyethylene urea (DHEU, 4,5-dihydroxy-2-imidazolidinone), dimethylolethylene urea (DMEU, 1,3-dihydroxymethyl-2-imidazolidinone), and dimethyldihydroxyethylene urea (DDI, 4,5-dihydroxy-1,3-dimethyl-2-imidazolidinone).

Suitable crosslinking agents include dialdehydes such as C₂-C₈ dialdehydes (e.g., glyoxal), C₂-C₈ dialdehyde acid analogs having at least one aldehyde group, and oligomers of these aldehyde and dialdehyde acid analogs, as described in U.S. Pat. Nos. 4,822,453; 4,888,093; 4,889,595; 4,889,596; 4,889,597; and 4,898,642. Other suitable dialdehyde crosslinking agents include those described in U.S. Pat. Nos. 4,853,086; 4,900,324; and 5,843,061.

Other suitable crosslinking agents include aldehyde and urea-based formaldehyde addition products. See, for example, U.S. Pat. Nos. 3,224,926; 3,241,533; 3,932,209; 4,035,147; 3,756,913; 4,689,118; 4,822,453; 3,440,135; 4,935,022; 3,819,470; and 3,658,613.

Suitable crosslinking agents include glyoxal adducts of ureas, for example, U.S. Pat. No. 4,968,774, and glyoxal/cyclic urea adducts as described in U.S. Pat. Nos. 4,285,690; 4,332,586; 4,396,391; 4,455,416; and 4,505,712.

Other suitable crosslinking agents include carboxylic acid crosslinking agents such as polycarboxylic acids. Polycarboxylic acid crosslinking agents (e.g., citric acid, propane tricarboxylic acid, and butane tetracarboxylic acid) and catalysts are described in U.S. Pat. Nos. 3,526,048; 4,820,307; 4,936,865; 4,975,209; and 5,221,285. The use of C₂-C₉ polycarboxylic acids that contain at least three carboxyl groups (e.g., citric acid and oxydisuccinic acid) as crosslinking agents is described in U.S. Pat. Nos. 5,137,537; 5,183,707; 5,190,563; 5,562,740, and 5,873,979.

Polymeric polycarboxylic acids are also suitable crosslinking agents. Suitable polymeric polycarboxylic acid crosslinking agents are described in U.S. Pat. Nos. 4,391,878; 4,420,368; 4,431,481; 5,049,235; 5,160,789; 5,442,899; 5,698,074; 5,496,476; 5,496,477; 5,728,771; 5,705,475; and 5,981,739. Polyacrylic acid and related copolymers as crosslinking agents are described U.S. Pat. Nos. 6,306,251; 5,549,791; and 5,998,511. Polymaleic acid crosslinking agents are described in U.S. Pat. No. 5,998,511.

Specific suitable polycarboxylic acid crosslinking agents include citric acid, tartaric acid, malic acid, succinic acid, glutaric acid, citraconic acid, itaconic acid, tartrate monosuccinic acid, maleic acid, polyacrylic acid, polymethacrylic acid, polymaleic acid, polymethylvinylether-co-maleate copolymer, polymethylvinylether-co-itaconate copolymer, copolymers of acrylic acid, and copolymers of maleic acid.

Other suitable crosslinking agents are described in U.S. Pat. Nos. 5,225,047; 5,366,591; 5,556,976; and 5,536,369.

Suitable catalysts can include acidic salts, such as ammonium chloride, ammonium sulfate, aluminum chloride, magnesium chloride, magnesium nitrate, and alkali metal salts of phosphorous-containing acids. In one embodiment, the crosslinking catalyst is sodium hypophosphite.

Mixtures or blends of crosslinking agents and catalysts can also be used.

The crosslinking agent is applied to the cellulosic fibers in an amount sufficient to effect interfiber crosslinking as described above. The amount applied to the cellulosic fibers can be from about 1 to about 10 percent by weight based on the total weight of fibers. In one embodiment, crosslinking agent in an amount from about 4 to about 6 percent by weight based on the total weight of fibers.

Suitable cellulosic fibers for forming the product of the invention include those known to those skilled in the art and include any fiber or fibrous mixture that can be crosslinked and from which a fibrous web or sheet can be formed.

Although available from other sources, cellulosic fibers are derived primarily from wood pulp. Suitable wood pulp fibers for use with the invention can be obtained from well-known chemical processes such as the kraft and sulfite processes, with or without subsequent bleaching. Pulp fibers can also be processed by thermomechanical, chemithermomechanical methods, or combinations thereof. The preferred pulp fiber is produced by chemical methods. Groundwood fibers, recycled or secondary wood pulp fibers, and bleached and unbleached wood pulp fibers can be used. Softwoods and hardwoods can be used. Details of the selection of wood pulp fibers are well known to those skilled in the art. These fibers are commercially available from a number of companies, including Weyerhaeuser Company, the assignee of the present invention. For example, suitable cellulose fibers produced from southern pine that are usable with the present invention are available from Weyerhaeuser Company under the designations CF416, NF405, PL416, FR516, and NB416.

The wood pulp fibers useful in the present invention can also be pretreated prior to use. This pretreatment may include physical treatment, such as subjecting the fibers to steam, or chemical treatment.

Although not to be construed as a limitation, examples of pretreating fibers include the application of surfactants or other liquids, which modify the surface chemistry of the fibers. Other pretreatments include incorporation of antimicrobials, pigments, dyes and densification or softening agents. Fibers pretreated with other chemicals, such as thermoplastic and thermosetting resins also may be used. Combinations of pretreatments also may be employed. Similar treatments can also be applied after formation of the fibrous product in post-treatment processes.

Cellulosic fibers treated with particle binders and/or densification/softness aids known in the art can also be employed in accordance with the present invention. The particle binders serve to attach other materials, such as superabsorbent polymers, as well as others, to the cellulosic fibers. Cellulosic fibers treated with suitable particle binders and/or densification/softness aids and the process for combining them with cellulose fibers are disclosed in the following U.S. patents: (1) U.S. Pat. No. 5,543,215, entitled “Polymeric Binders for Binding Particles to Fibers”; (2) U.S. Pat. No. 5,538,783, entitled “Non-Polymeric Organic Binders for Binding Particles to Fibers”; (3) U.S. Pat. No. 5,300,192, entitled “Wet Laid Fiber Sheet Manufacturing With Reactivatable Binders for Binding Particles to Binders”; (4) U.S. Pat. No. 5,352,480, entitled “Method for Binding Particles to Fibers Using Reactivatable Binders”; (5) U.S. Pat. No. 5,308,896, entitled “Particle Binders for High-Bulk Fibers”; (6) U.S. Pat. No. 5,589,256, entitled “Particle Binders that Enhance Fiber Densification”; (7) U.S. Pat. No. 5,672,418, entitled “Particle Binders”; (8) U.S. Pat. No. 5,607,759, entitled “Particle Binding to Fibers”; (9) U.S. Pat. No. 5,693,411, entitled “Binders for Binding Water Soluble Particles to Fibers”; (10) U.S. Pat. No.5,547,745, entitled “Particle Binders”; (11) U.S. Pat. No.5,641,561, entitled “Particle Binding to Fibers”; (12) U.S. Pat. No. 5,308,896, entitled “Particle Binders for High-Bulk Fibers”; (13) U.S. Pat. No. 5,498,478, entitled “Polyethylene Glycol as a Binder Material for Fibers”; (14) U.S. Pat. No. 5,609,727, entitled “Fibrous Product for Binding Particles”; (15) U.S. Pat. No. 5,571,618, entitled “Reactivatable Binders for Binding Particles to Fibers”; (16) U.S. Pat. No. 5,447,977, entitled “Particle Binders for High Bulk Fibers”; (17) U.S. Pat. No. 5,614,570, entitled “Absorbent Articles Containing Binder Carrying High Bulk Fibers”; (18) U.S. Pat. No. 5,789,326, entitled “Binder Treated Fibers”; and (19) U.S. Pat. No. 5,611,885, entitled “Particle Binders”, each expressly incorporated herein by reference.

In addition to natural fibers, synthetic fibers including polymeric fibers, such as polyolefin, polyamide, polyester, polyvinyl alcohol, polyvinyl acetate fibers, can also be incorporated into the product. Suitable synthetic fibers include, for example, polyethylene terephthalate, polyethylene, polypropylene, nylon, and rayon fibers. Other suitable synthetic fibers include those made from thermoplastic polymers, cellulosic and other fibers coated with thermoplastic polymers, and multicomponent fibers in which at least one of the components includes a thermoplastic polymer. Single and multicomponent fibers can be manufactured from polyester, polyethylene, polypropylene, and other conventional thermoplastic fibrous materials. Single and multicomponent fibers are commercially available. Suitable bicomponent fibers include CELBOND fibers available from Hoechst-Celanese Company. The product can also include combinations of natural and synthetic fibers.

In one embodiment, the crosslinked cellulosic fiber is a citric acid crosslinked fiber.

The product of the invention is formed by treating crosslinked fibers with a bonding agent followed by heating to effect bonding between fibers (i.e., interfiber bonding). The bonding agent serves to enhance the structural integrity of the product. Suitable bonding agents include thermoplastic materials, such as bicomponent fibers and latexes, and wet strength agents.

When the bonding agent is a thermoplastic fiber, the fiber can be combined with cellulosic fibers and then formed into the web to be subsequently treated. When the bonding agent is a wet strength agent, the bonding agent can be applied to the web prior to subjecting the web to fiber bonding conditions.

Suitable thermoplastic fibers include cellulosic and other fibers coated with thermoplastic polymers, and multicomponent fibers in which at least one of the components includes a thermoplastic polymer. Single and multicomponent fibers can be manufactured from polyester, polyethylene, polypropylene, and other conventional thermoplastic fibrous materials. Single and multicomponent fibers are commercially available. Suitable bicomponent fibers include CELBOND fibers available from Hoechst-Celanese Company.

Suitable wet strength agents include cationic modified starch having nitrogen-containing groups (e.g., amino groups) such as those available from National Starch and Chemical Corp., Bridgewater, N.J.; latex; wet strength resins, such as polyamide-epichlorohydrin resin (e.g., KYMENE 557LX, Hercules, Inc., Wilmington, Del.), and polyacrylamide resin (see, e.g., U.S. Pat. No. 3,556,932 and also the commercially available polyacrylamide marketed by American Cyanamid Co., Stanford, Conn., under the trade name PAREZ 631 NC); urea formaldehyde and melamine formaldehyde resins; and polyethylenimine resins. A general discussion on wet strength resins utilized in the paper field, and generally applicable in the present invention, can be found in TAPPI monograph series No. 29, “Wet Strength in Paper and Paperboard”, Technical Association of the Pulp and Paper Industry (New York, 1965).

In one embodiment, the bonding agent is a latex. Suitable latexes include a latex designated PD-8161 commercially available from H.B. Fuller.

In another embodiment, the product by treating crosslinked cellulosic fibers with a latex and a latex fixative. Suitable latex fixatives include a fixative designated CARTAFIX-U commercially available from Clariant Corp.

In one embodiment, the product is formed by treating crosslinked cellulosic fibers with about 2 percent by weight latex and about 10 percent by weight fixative based on the total weight of latex.

In other embodiments, the product can include other fibers. Other fibers include, for example, the cellulosic fibers, particularly the wood pulp fibers described above, as well as hemp, bagasse, cotton, groundwood, bleached and unbleached pulp, recycled or secondary fibers.

In another aspect of the invention, methods for forming the bonded cellulosic fibrous product are provided. As noted above, the product can be formed by airlaid or extrusion processes.

As described above, the product of the invention is formed by subjecting a web that includes crosslinked cellulosic fibers and a bonding agent to a temperature and for a time sufficient to effect interfiber bonding. The bonding can be performed by several methods. In one embodiment, the product is formed by heating in an oven in which high temperature and large volumes of air are drawn through the web. In another embodiment, bonding takes place after the webs have been placed in boxes for shipping. In this embodiment, boxes containing the treated webs are passed through a dryer (e.g., a kiln dryer) to complete the crosslinking reaction.

The product of the present invention can be formed as an extended web or sheet that has structural integrity and sheet strength sufficient to permit the fibrous web to be rolled, transported, and used in rolled form in subsequent processes.

The product of the present invention can be supplied in a fibrous rolled form and readily incorporated into subsequent processes. The product can be advantageously incorporated into a variety of absorbent articles, such as diapers, including disposable diapers and training pants; feminine care products, including sanitary napkins, tampons, and pant liners; adult incontinence products; toweling; surgical and dental sponges; bandages; food tray pads; and the like.

In a further aspect, the present invention provides absorbent articles that include the bonded cellulosic fibrous product. The product can be combined with one or more other layers to provide structures that can be incorporated into absorbent articles such as infant diapers, adult incontinence products, and feminine care products.

The compression recovery for representative products of the invention under 0.5 psi load for one week measured 24 hours after load removal is summarized in Table 1. In Table 1, initial density, density after one week at 0.5 psi load (density after load), and density 24 hours after removal of load (recovered density) are reported in units of g/cc. In the table, XLA refers to citric acid crosslinked cellulose fibers, T105 refers to bicomponent binding fibers (CELBOND T105), PD8161 refers to a latex (H.B. Fuller), and U refers to a binding fiber (Unitika T224).

TABLE 1
Compression Recovery Summary.

| Product | Initial | | |
|---|---|---|---|
| Composition | Density | Density After Load | Recovered Density |
| 100% XLA | 0.048 | 0.061 | 0.058 |
| 90 XLA/10 T105^{a} | 0.044 | 0.059 | 0.055 |
| 90 XLA/10 T105^{b} | 0.044 | 0.060 | 0.055 |
| 90 XLA/10 T105^{c} | 0.045 | 0.060 | 0.056 |
| 90 XLA/10 T105^{d} | 0.047 | 0.059 | 0.056 |
| 98 XLA/2 PD8161 | 0.056 | 0.064 | 0.060 |
| 98 XLA/2 PD8161^{e} | 0.045 | 0.056 | 0.052 |
| 95 XLA/5 T105 | 0.044 | 0.060 | 0.055 |
| 90 XLA/10 T105 | 0.036 | 0.050 | 0.046 |
| 90 XLA/8T105/2U | 0.042 | 0.058 | 0.055 |

^{a}Foam-formed product formed using surfactant RW150.
^{b}Foam-formed product formed using surfactant Inc30.
^{c}Product includes wet strength agent (KYMENE, 40 pounds per ton fiber).
^{d}Product includes wet strength agent (PAREZ, 40 pounds per ton fiber).
^{e}Product includes latex fixative (CARTAFIX U).

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method for making a cellulosic fibrous product, comprising:
(a) forming a fibrous web comprising crosslinked cellulosic fibers;
(b) treating the web with a bonding agent; and
(c) heating the web to effect bonding between the fibers to provide a bonded cellulosic fibrous product having a density of from about 0.02 to about 0.06 g/cm³.

2. The method of claim 1, wherein the bonding agent is at least one of a latex and a wet strength agent.

3. The method of claim 1, wherein the bonding agent comprises a latex.

4. The method of claim 3, wherein the bonding agent further comprises a latex fixative.

5. The method of claim 1, wherein the bonding agent comprises a polyamide-epichlorohydrin resin.

6. The method of claim 1, wherein the bonding agent comprises a polyacrylamide resin.

7. The method of claim 1, wherein the fibrous web further comprises other cellulosic fibers.

8. The method of claim 1, wherein the fibrous web is at least one of an airlaid web and an extruded web.

9. A method for making a cellulosic fibrous product, comprising:
(a) forming a fibrous web comprising crosslinked cellulosic fibers and thermoplastic fibers; and
(b) heating the web to effect bonding between the fibers to provide a bonded cellulosic fibrous product having a density of from about 0.02 to about 0.06 g/cm³.

10. The method of claim 9, wherein the thermoplastic fibers comprises bicomponent fibers.

11. The method of claim 9, wherein the fibrous web further comprises other cellulosic fibers.

12. The method of claim 9, wherein the fibrous web is at least one of an airlaid web and an extruded web.

13. A bonded cellulosic fibrous product, comprising crosslinked cellulosic fibers treated with an amount of a bonding agent effective to provide a bonded cellulosic fibrous product having a density of from about 0.02 to about 0.06 g/cm³.

14. The product of claim 13, wherein the product returns to from about 75 to about 100 percent of its original density after being densified to from about 0.075 to about 0.2 g/cm³.

15. The product of claim 13 wherein the product returns to from about 75 to about 100 percent of its original density after 7 days at 0.5 psi.

16. The product of claim 13 wherein the bonding agent is at least one of a latex, a wet strength agent, and a thermoplastic fiber.

17. The product of claim 13, wherein the bonding agent comprises a latex.

18. The product of claim 17, wherein the bonding agent further comprises a latex fixative.

19. The product of claim 13, wherein the bonding agent comprises a polyamide-epichlorohydrin resin.

20. The product of claim 13, wherein the bonding agent comprises a polyacrylamide resin.

21. The product of claim 13, wherein the bonding agent comprises a bicomponent fiber.

22. The product of claim 13 further comprising other cellulosic fibers.

23. The product of claim 13, wherein the product is at least one of an airlaid product and an extruded product.

24. A bonded cellulosic fibrous product, comprising crosslinked cellulosic fibers treated with an amount of a latex effective to provide a bonded cellulosic fibrous product having a density of from about 0.02 to about 0.06 g/cm³.

25. The product of claim 24, wherein the product returns to from about 75 to about 100 percent of its original density after being densified to from about 0.075 to about 0.2 g/cm³.

26. The product of claim 24, wherein the product returns to from about 75 to about 100 percent of its original density after 7 days at 0.5 psi.

27. The product of claim 24 further comprising other cellulosic fibers.

28. The product of claim 24, wherein the product is at least one of an airlaid product and an extruded product.

29. A bonded cellulosic fibrous product, comprising crosslinked cellulosic fibers and an amount of thermoplastic fibers effective to provide a bonded cellulosic fibrous product having a density of from about 0.02 to about 0.06 g/cm³.

30. The product of claim 29, wherein the product returns to from about 75 to about 100 percent of its original density after being densified to from about 0.075 to about 0.2 g/cm³.

31. The product of claim 29, wherein the product returns to from about 75 to about 100 percent of its original density after 7 days at 0.5 psi.

32. The product of claim 29 further comprising other cellulosic fibers.

33. The product of claim 29, wherein the product is at least one of an airlaid product and an extruded product.

34. A personal care absorbent product comprising the product of any one of claims 13, 24, or 29.

35. The product of claim 34, wherein the product is at least one of an infant diaper, adult incontinence product, and a feminine hygiene product.
